(19) United States
(12) Patent Application Publication
(10) Pub. No.: US 2011/0111473 A1
(43) Pub. Date: May 12, 2011

(54) YEAST CELLS AND METHODS FOR INCREASING ETHANOL PRODUCTION

(75) Inventor: Joy Doran Peterson, Athens, GA (US)

(73) Assignee: University of Georgia Research Foundation, Inc, Athens, GA (US)

(21) Appl. No.: 12/990,031

(22) PCT Filed: May 8, 2009
(86) PCT No.: PCT/US09/43358

§ 371 Date: Jan. 3, 2011

Related U.S. Application Data
(60) Provisional application No. 61/051,851, filed on May 9, 2008.

(51) Int. Cl. C12P 7/06; C12N 1/16

(52) U.S. Cl. 435/161; 435/255.1

(57) ABSTRACT

Provided herein are methods for producing ethanol using yeast to ferment pretreated solid lignocellulosic materials such as pretreated solid lignocellulosic materials obtained from softwoods like pine. The pretreated solid lignocellulosic material is present at a concentration of at least 12% solids, and at least 30 grams ethanol per liter, preferably at least 40 grams ethanol per liter, are produced within 48 hours. In some aspects, the pretreated solid lignocellulosic material is not detoxified prior to the fermentation. Also provided herein are yeast that ferment a composition that includes pretreated solid lignocellulosic material at a concentration of at least 12% solids to yield at least 30 grams ethanol per liter, preferably at least 40 grams ethanol per liter, in 48 hours. Further provided herein are methods for acclimatizing yeast.

This application claims the benefit of U.S. Provisional Application Ser. No. 61/051,851, filed May 9, 2008, which is incorporated by reference herein.

### BACKGROUND

Fermenting sugars produces ethanol. The sugars can be derived from a variety of sources. In Brazil, sugar from sugar cane is the primary feedstock for the huge Brazilian industry. In North America, the sugar is usually derived from the enzymatic hydrolysis (the conversion of starch to sugar) of starch containing crops such as corn or wheat. The enzymatic hydrolysis of starch is a cheap, simple, and effective process. This well developed process sets the baseline that other hydrolysis processes are compared against. The drawback to producing ethanol from sugar or starch is that the feedstock tends to be expensive and widely used for other applications.

Lignocellulosic materials such as agricultural, hardwood and softwood residues are potential sources of sugars for ethanol production. The cellulose and hemicellulose components of these materials are essentially long, molecular chains of sugars. They are protected by lignin, which is the glue that holds all of this material together. The technological hurdles that are presented by the materials are:
The separation of lignin from the cellulose and hemicellulose to make the material susceptible to hydrolysis.
The hydrolysis of cellulose and hemicellulose takes place at different rates and over reaction can degrade the sugars into materials that are not suitable for ethanol production.
The hydrolysis of these materials produces a variety of sugars. Not all of these sugars are fermentable with the standard yeast that is used in the grain ethanol industry. The pentose sugars are particularly difficult to ferment.

Agricultural residues and hardwoods are similar in that they have a lower lignin content and the hemicellulose produces significant amounts of pentose sugars. Softwood is a term used for wood from conifers. Well-known softwood-producing trees include pine, spruce, fir, larch, douglas-fir and hemlock. The term softwood is also used for the trees themselves. Softwoods have a higher lignin content, which makes the hydrolysis step more difficult, but they generally produce less pentose sugars. Softwood is the primary source of lignocellulosic biomass in the northern hemisphere and contains about 44% cellulose, 22% hemicellulose and 28% lignin (Galbe and Zacchi, 2002, Appl. Microbiol. Biotechnol., 59:618). Recently, softwoods have been the subject of great interest in Sweden, Canada and the United States as a renewable source for ethanol production. It was estimated in 2002 that by 2020 there should be sufficient amounts of woody biomass for the production of bioethanol to be able to replace more than 20% of the gasoline and diesel used at that time (Galbe and Zacchi, 2002, Appl. Microbiol. Biotechnol., 59:618).

The general requirements for an organism to be used in ethanol production is that it should give a high ethanol yield, a high productivity and be able to withstand high ethanol concentrations in order to keep distillation costs low (Olofsson et al., 2008, Biotechnol. Biofuels, 1:7). In addition to these general requirements, inhibitor tolerance, temperature tolerance and the ability to utilize multiple sugars are essential for simultaneous saccharification and fermentation (SSF) applications. Tolerance towards low pH-values will minimize the risk of contamination. The most frequently used microorganism for fermenting ethanol in industrial processes is the common Bakers' yeast, Saccharomyces cerevisiae (Galbe and Zacchi, 2002, Appl. Microbiol. Biotechnol., 59:618). This organism produces ethanol at a high yield (higher than 0.45 g eat optimal conditions), a high specific rate (up to 1.3 g g⁻¹ cell mass h⁻¹) and a very high ethanol tolerance, over 100 g L⁻¹ has been reported for some strains and media (Olofsson et al., 2008, Biotechnol. Biofuels, 1:7). In addition, the organism has proven to be robust to other inhibitors, and hence it is suitable for fermentation of lignocellulosic materials. Zymomonas mobilis can ferment glucose to ethanol with higher yields, but is less robust. S. cerevisiae has a great advantage over Z. mobilis in the fermentation of softwood hydrolysates as S. cerevisiae ferments mannose and, after adaptation, also galactose (Galbe and Zacchi, 2002, Appl. Microbiol. Biotechnol., 59:618). The thermophilic bacterial species Clostridium, Thermoanaerobium, and Thermoanaerobacterium are able to generate high ethanol yields, but have a very poor tolerance to ethanol (Keating et al., 2004).

Various strains of S. cerevisiae have certain advantages over Baker's yeast. One such strain, Tembec T1, is an industrially adapted yeast strain, which was isolated from a spent sulfite liquor (SSL) stream exiting the Tembec pulp and paper mill in Canada. It is a robust strain that is able to effectively convert lignocellulose-derived substrates to ethanol in the presence of toxic inhibitory compounds (Keating et al., 2004, J. Ind. Microbiol. Biotechnol. 31:235). Another yeast strain, S. cerevisiae Y-1528, is a natural isolate obtained from a culture collection that was selected on the basis of its ability to ferment galactose and utilize it as effectively as glucose and mannose (Keating et al., 2004, J. Ind. Microbiol. Biotechnol. 31:235). In a study comparing the performance of Tembec T1 and Y-1528 strains used individually, or in combination, Y-1528 clearly out-performed Tembec T1 on the three different lignocellulosic substrates used (Keating et al., 2004, J. Ind. Microbiol. Biotechnol. 31:235). There was some evidence of synergistic interaction between the strains.

Two S. cerevisiae strains, ATCC 211239 and NRRL Y-12632, and one Pichia stipitis strain, NRRL Y-7124, were evaluated for their response over time to furfural and HMF at a range of concentrations, separately and combined (Liu et al., 2004, J. Ind. Microbiol. Biotechnol., 31:345). As expected, cell growth in all three yeast strains was dose-dependently inhibited by both furfural and HMF. After a prolonged lag-phase, however, cell growth recovered and glucose consumption and ethanol production reached the maximum extent. This suggests that the cells underwent a major shift in their physiology in adapting to the chemical stress, and that such adaptation can be used to produce yeast strains that are tolerant to the effects of inhibitors such as furfural and HMF. The effectiveness of such adaptation was also seen is a study comparing SSF with Baker's yeast, adapted Baker's yeast and TMB3000, which is an inhibitor-tolerant S. cerevisiae strain isolated from SSL from a plant in Sweden (Alkasrawi et al., 2006, Enzyme Microb. Tech., 38:279). Baker's yeast was adapted by cultivation on the hemicellulose hydrolysate obtained in the pretreatment of softwood and had the highest ethanol productivity at all substrate loads. It even performed better than TMB3000, and both adapted Baker's yeast and TMB3000 were able to convert galactose, with adapted Baker's yeast being more effective.

Since none of the available yeasts and bacteria are able to satisfy all the technical criteria, and since lignocellulose hydrolysates contain pentoses, which are not readily fermented by the abovementioned organisms, there have been attempts to genetically engineer S. cerevisiae, Z mobilis and the bacteria E. coli. One example is recombinant E. coli containing both pdc and adhB genes derived from Z. mobilis which can ferment both glucose and xylose to ethanol (Qian et al., 2006, Appl. Biochem. Biotechnol., 134:273). HMF inhibition is overcome in S. cerevisiae strains that are able to reduce HMF to 5-hydroxymethylfurfuryl alcohol and this reduction is NADPH- and NADH-coupled (Nilsson et al., 2005, Appl. Environ. Microbiol., 71:7866). In a study comparing the performance of six selected S. cerevisiae strains (Baker's yeast, CBS 8066, CEN/PK 113-7D, TMB3000, TMB3500 and USM21), TMB3000 was by far the most efficient strain and was the only strain showing a significant constitutive NADH-coupled in vitro reduction of HMF (Modig et al., 2008, Biotechnol. Bioeng., 100:423). Microarray analyses were performed on two yeast strains, TMB3000 and a laboratory strain, CBS8066, using genome-wide transcription analysis of known yeast reductase and dehydrogenase genes. Candidate genes were cloned and then over-expressed in a CEN.PK strain that does not itself show tolerance to HMF (Petersson et al., 2006, Yeast, 23:455). Over-expression of the ADH6 gene, which encodes a strictly NADPH-dependent alcohol dehydrogenase, resulted in a three-fold higher specific HMF uptake rate than the reference strain under anaerobic conditions, a four-fold increase under aerobic conditions, and high HMF reducing activity with NADPH as a co-factor. Improved tolerance to furfural could also be achieved by genetically engineering the co-factor balance inside the cell. S. cerevisiae gene deletion mutants of the pentose phosphate pathway (PPP) genes zwf1, gnd1, rpe1 and tkl1 had inhibited growth in the presence of furfural, indicating that S. cerevisiae's tolerance to furfural is associated with the activity of PPP, which is the main source of cytoplasmic NADPH. Over-expression of the ZWF1 gene encoding glucose-6 phosphate dehydrogenase enabled growth at furfural concentrations that are normally toxic to S. cerevisiae (Almeida et al., 2007, J. Chem. Technol. Biotechnol., 82:340). The design of a genetically engineered S. cerevisiae strain tolerant to phenolic derivatives has also been explored. Over-expression of the PAD1 gene encoding phenylacrylic acid decarboxylase in S. cerevisiae resulted in a 50-100% improvement in ethanol productivity when the cells were cultured in the presence of phenolic derivatives (Almeida et al., 2007, J. Chem. Technol. Biotechnol., 82:340). Targeted metabolic engineering appears to be a promising approach for the development of efficient lignocellulose-based ethanol fermentation.

Co-culture can also be a way of overcoming the limitations of each strain. S. cerevisia 2.535 cannot ferment xylose to ethanol although it can ferment glucose to ethanol and metabolize and/or tolerate some inhibitors at low concentrations. Pachysolen tannophilis ATCC 2.1662 can ferment both glucose and xylose, but is sensitive to inhibitors. Recombinant E. coli (containing pdc and adhB genes as described above) can metabolize both glucose and xylose. Co-culture of S. cerevisia 2.535 with P. tannophilis ATCC 2.1662 (1) and that of S. cerevisia 2.535 with recombinant E. coli(2) was used with and without adaptation over 5 batches of culture (Qian et al., 2006, Appl. Biochem. Biotechnol., 134:273). Adaptation increased ethanol concentrations by 50% for co-culture 1 and by 120% for co-culture 2. Both inhibitor tolerance and fermentation ability of the microorganisms were substantially enhanced by adaptation and the fermentation performance of both co-cultures was similar, and better than when these microorganisms are used alone.

### SUMMARY OF THE INVENTION

Provided herein are methods for producing ethanol. The methods may include fermenting a composition with a yeast, wherein the composition includes pretreated solid lignocellulosic material, the pretreated solid lignocellulosic material is present at a concentration of at least 12% solids, and at least 30 grams ethanol per liter are produced. The at least 30 grams ethanol per liter are produced within 48 hours. In some aspects, at least 40 grams ethanol per liter are produced, for instance, within 48 hours. The fermenting may include the addition of at least 0.02 and no greater than 2 grams yeast cells per liter, and may be a batch simultaneous saccharification and fermentation. In some aspects, the pretreated solid lignocellulosic material is present at a concentration of no greater than 17.5% solids.

The pretreated solid lignocellulosic material may be detoxified before the yeast is cultured, and in some aspects the pretreated solid lignocellulosic material is not detoxified prior to the culturing. The composition may include furfural, 5-hydroxymethylfurfural, formic acid, acetic acid, vanillic acid, vanillin, levulinic acid, or a combination thereof. The furfural may be present at a concentration of at least 700 mg/L or at least 900 mg/L. The 5-hydroxymethylfurfural may be present at a concentration of at least 800 mg/L or at least 1200 mg/L. The formic acid may be present at a concentration of at least 200 mg/L, or at least 400 mg/L. The acetic acid may be present at a concentration of at least 500 mg/L, or at least 900 mg/L. The vanillic acid may be present at a concentration of at least 7.5 mg/L, or at least 12 mg/L. The vanillin may be present at a concentration of at least 20 mg/L, or at least 60 mg/L. The levulinic acid may be present at a concentration of at least 50 mg/L, or at least 130 mg/L.

The pretreated solid lignocellulosic material is pine, such as Pinus taeda. Prior to the fermenting the yeast may be revived from a frozen stock. Reviving may include transferring a frozen stock directly to a reviving composition that includes the pretreated solid lignocellulosic material. A reviving composition may include the pretreated solid lignocellulosic material at a concentration of at least 6%. The yeast may be Saccharomyces cerevisiae, such as AJP50.

Also provided herein are yeast that ferment a composition that includes pretreated solid lignocellulosic material at a concentration of at least 12% solids to yield at least 30 grams ethanol per liter, preferably at least 40 grams ethanol per liter, in 48 hours. In some aspects, the pretreated solid lignocellulosic material that are fermented is not detoxified. The composition may include furfural, 5-hydroxymethylfurfural, formic acid, acetic acid, vanillic acid, vanillin, levulinic acid, or a combination thereof. The furfural may be present at a concentration of at least 700 mg/L or at least 900 mg/L. The 5-hydroxymethylfurfural may be present at a concentration of at least 800 mg/L or at least 1200 mg/L. The formic acid may be present at a concentration of at least 200 mg/L, or at least 400 mg/L. The acetic acid may be present at a concentration of at least 500 mg/L, or at least 900 mg/L. The vanillic acid may be present at a concentration of at least 7.5 mg/L, or at least 12 mg/L. The vanillin may be present at a concentration of at least 20 mg/L, or at least 60 mg/L. The levulinic acid may be present at a concentration of at least 50 mg/L, or at least 130 mg/L. The yeast may have the characteristics of AJP50.

Further provided herein are methods for acclimatizing yeast. The methods may include culturing yeast in a first composition that includes pretreated solid lignocellulosic material, transferring a sample of the cultured yeast to a second composition that includes pretreated solid lignocellulosic material and culturing the yeast in the second composition, and repeating the transferring until the yeast will produce at least 30 grams ethanol per liter during a batch simultaneous saccharification and fermentation with at least 12% solids. The pretreated solid lignocellulosic material in the first composition may be present at a concentration of at least 5% solids. The second composition may include the pretreated solid lignocellulosic material at a concentration greater than the first composition. The

repeated transferring may include transferring the yeast to compositions having the pretreated solid lignocellulosic material at increasing concentrations of solids.

In some aspects, the pretreated solid lignocellulosic material that are used in the methods is not detoxified. The composition may include furfural, 5-hydroxymethylfurfural, formic acid, acetic acid, vanillic acid, vanillin, levulinic acid, or a combination thereof. The furfural may be present at a concentration of at least 700 mg/L or at least 900 mg/L. The 5-hydroxymethylfurfural may be present at a concentration of at least 800 mg/L or at least 1200 mg/L. The formic acid may be present at a concentration of at least 200 mg/L, or at least 400 mg/L. The acetic acid may be present at a concentration of at least 500 mg/L, or at least 900 mg/L. The vanillic acid may be present at a concentration of at least 7.5 mg/L, or at least 12 mg/L. The vanillin may be present at a concentration of at least 20 mg/L, or at least 60 mg/L.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Ethanol production from varying concentrations of G4.

FIG. 2. Effect of yeast acclimatization on ethanol production from pretreated G3S2 at 17.5% dry weight solids treated with enzymes Novozyme 13 and Cellobiase at a conc. of 15 FPU/g and 60 U/g dry weight of total solids.

FIG. 3. SSF ethanol production using a low inoculum level in 3.3% sulfur dioxide pretreated pine.

FIG. 4. Ethanol production from 3.5% sulfur dioxide pretreated pine, comparison of ADY and AJP50.

FIG. 5. Schematic of steps for scale up of fermentations.

### DETAILED DESCRIPTION OF ILLUSTRATIVE EMBODIMENTS

The present invention includes yeast strains for fermenting lignocellulosic materials into fermentation products. The term “fermentation product” means a product produced by a process including a fermentation step using a fermenting organism.

Fermentation products contemplated according to the invention include alcohols such as, but not limited to, ethanol. Ethanol obtained according to the methods described herein may be used as fuel ethanol, as drinking ethanol, e.g., potable ethanol, or industrial ethanol. A yeast of the present invention may be isolated. A yeast is “isolated” when it has been removed from its natural environment and can be grown as a pure culture.

A yeast strain of the present invention may be produced by acclimatizing the yeast to growth in medium that contains fermentable sugars, toxic compounds that can inhibit the growth of yeast, and solid lignocellulosic material. As used herein, “solid lignocellulosic material,” “lignocellulosic solids,” and “solids” refers to total dry weight of a pretreated lignocellulosic material. In general, a method for acclimatizing a yeast includes exposure of a yeast to a medium under conditions that promote fermentation and the production of an alcohol, such as ethanol.

The beginning of the acclimatization process may begin by incubating yeast in non-selective conditions, e.g., a complex medium that promotes growth of the yeast. An example of such a complex medium includes yeast extract (10 g/L), peptone (20 g/L), and dextrose (40 g/L). The first non-selective step may be in liquid medium or on a solid plate. Cells are grown to yield an amount that can be used to inoculate a volume of selective medium in a fermenter. When grown to inoculate a fermenter for acclimatization, the yeast are typically grown in non-selective liquid medium until the culture has enough cells for an inoculum of an optical density at 600 nanometers of 0.5 into a fermenter containing selective medium.

Any yeast may be used in this method, and commercially available examples of suitable yeast include, e.g., RED STAR and ETHANOL RED yeast (available from Fermentis/Lesaffre, USA), FALI (available from Fleischmann's Yeast, USA), SUPERSTART and THERMOSACC fresh yeast (available from Ethanol Technology, Wis., USA), BIOFERM AFT and XR (available from NABC—North American Bioproducts Corporation, GA, USA), GERT STRAND (available from Gert Strand AB, Sweden), and FERMIOL (available from DSM Specialties). Genetically modified yeast may be used, including those capable of converting hexoses and pentoses to ethanol.

A selective medium may include pretreated lignocellulosic solids. Typically, the lignocellulosic solids used in a selection procedure are from a hardwood, or a softwood such as, but not limited to, pine (including Loblolly pine, Pinus taeda), spruce, Douglas fir, longleaf pine, bald cypress, tamarack, larch, and yew. The selective medium typically contains one or more toxic compounds that can inhibit the growth of yeast. Such toxic compounds include, but are not limited to, furfural, 5-hydroxymethylfurfural (HMF), levulinic acid, acetic acid, formic acid, uronic acid, 4-hydroxybenzoic acid, vanillic acid, vanillin, phenol, cinnamaldehyde, formaldehyde 2-furoic acid, 3,4-dihydrxybenzoic acid, 3,4-dihydroxybenzaldehyde, salicylic acid, homovanillic acid, benzoic acid, and ferulic acid. Preferably, a selective medium contains furfural, HMF, formic acid, acetic acid or a combination thereof. The concentration of furfural may be, for instance, at least 700 mg/L or at least 900 mg/L, the concentration of HMF may be, for instance, at least 800 mg/L or at least 1200 mg/L, the concentration of formic acid may be, for instance, at least 200 mg/L or at least 400 mg/L, the concentration of acetic acid may be, for instance, at least 500 mg/L or at least 900 mg/L, the concentration of vanillic acid may be, for instance, at least 7.5 mg/L or at least 12 mg/L, the concentration of vanillin may be, for instance, at least 20 mg/L or at least 60 mg/L, the concentration of levulinic acid may be, for instance, at least 50 mg/L or at least 130 mg/L.

Pretreatment of lignocellulosic material may be accomplished with methods known in the art and routinely used. Pretreatment refers to the solubilization and separation of one or more of the four major components of biomass—hemicellulose, cellulose, lignin, and extractives—to make the remaining solid biomass more accessible to further chemical or biological treatment. Typically, lignocellulosic material is physically pretreated to increase the surface area of the material, for instance by chipping to a particle size that is 10 mm or less. In one pretreatment, a known amount of chips may be treated with SO₂ at 0.5% to 5%, such as 3% or 4% SO₂ (wt/wt), at a temperature of between 190° C. and 220° C., such as 213° C. or 215° C., for between 2 and 7 minutes, such as 5 minutes. The result is a single mixture that is not washed, with liquid and solid mixed together. Another pretreatment involves two steps. A known amount of chips may be treated with SO₂ at 0.5% to 5%, such as 2.5% SO₂ (wt/wt), at a temperature of between 190° C. and 220° C., such as 190° C., for between 2 and 7 minutes, such as 5 minutes. The resulting material may be pressed to collect liquid. The remaining solids may then be washed with water and pressed to a dry matter content of 40%. This material may be impregnated with SO₂, for instance, 2.5% SO₂ wt/wt of moisture content in the solids, at a temperature of between 190° C. and 220° C., such as 210° C., for between 2 and 7 minutes, such as 5 minutes. The result is a single mixture containing solids that have been washed. Other pretreatment methods are known in the art and may be used in the acclimatization methods described herein. Preferably, the medium resulting from the two step pretreatment method is used as a selective medium for acclimatization.

Any suitable fermenter can be used for the acclimatization. For instance, without intending to be limiting, a fermenter may have a volume of fermentation medium of, for instance, 100 mls, or a volume of, for instance, at least 2 liters. Selective medium is added to the appropriate volume and subjected to simultaneous saccharification and fermentation (SSF) using the yeast strain to be acclimatized. The selective medium added may be from either of the two pretreatments described above, preferably the medium resulting from the two step pretreatment method is used as a selective medium for acclimatization. The selective medium is added to result in a solids level of at least 5% up to 17.5% (dry weight solids per total volume) in increments of 0.5%, i.e., at least 5%, at least 5.5%, at least 6%, etc., up to 17.5%.

Typically, the fermentation medium also contains enzymes to promote the hydrolysis of complex carbohydrates (e.g., Novozyme 13 and Cellobiase) and additional complex medium, such as tryptic soy broth. An example of a tryptic soy broth is 15 grams Tryptone (Pancreatic Digest of Casein), 5 grams Soytone (Papaic Digest of Soybean Meal), and 5 grams Sodium Chloride per liter total fermenter volume. To maintain selective pressure on the cells the fermentation medium is typically not supplemented with fermentable sugars. In some aspects, the fermentation medium may be supplemented with fermentable sugars, such as glucose, at a concentration of no greater than 20 g/L, no greater than 10 g/L, no greater than 5 g/L, or no greater than 1 g/L. The cells prepared in complex non-selective medium are also added, and the fermentation allowed to proceed at 37° C. at a pH of 5. Ethanol production may be followed by collecting a sample at appropriate intervals, such as every 12 or 24 hours.

The SSF is allowed to proceed, and a sample may be removed from the SSF and transferred to a new SSF. Typically, a volume equivalent to 10% (vol/vol) may be transferred. Preferably, a transfer occurs after an SSF has fermented between 24 and 96 hours, preferably between 48 and 72 hours. In some cases it may be useful to begin the transfers at 72 hours and begin to decrease the time an SSF occurs before transfer, for instance, 48 hours. Initially, the selective medium is added to result in 5% solids, and the yeast transferred to fermenters containing increasing amounts of solids. For instance, the solids can increase in steps as follows: 7%, 9%, 12%, 13%, 14%, 15%, 16%, 17%, and 17.5%. Typically, the yeast is kept at a particular concentration of solids until it can be transferred to a higher concentration and survive in the new selective medium. Typically, yeast may be transferred at least 20 times, at least 30 times, at least 40 times, or at least 50 times before acclimatized yeast having one or more of the characteristics described herein are obtained. Typically, the selective medium is not detoxified prior to addition of yeast for acclimatization. Using this methodology, yeast were unexpectedly obtained that could reproduce and ferment the sugars to produce ethanol in an SSF containing 17.5% solids.

Yeast obtained may be stored by producing a glycerol stock culture. Yeast (10⁶ cells) may be suspended in 40% (w/v) glycerol stock culture and frozen at −80° C. When needed, the frozen cells may be revived as described herein.

Yeast of the present invention may have the characteristic of producing ethanol such that an ethanol yield from a fermentation is at least 82% up to 100% of theoretical in single integer increments, i.e., at least 82%, at least 83%, at least 84%, etc., up to 100% of theoretical. The phrase “ethanol yield” refers to the percentage of theoretical ethanol produced during a fermentation. The theoretical ethanol that can be produced during a fermentation is based on the amount of fermentable sugars present in the pretreated material. Unless stated otherwise, “fermentable sugars” refers to six carbon sugars that can be fermented by Saccharomyces yeast. Using the parameters described above for pretreatment of lignocellulosic material derived from pine, the percentage of fermentable sugars may be between 44% to 56% of the total dry matter of the pretreated material. As is known to the person skilled in the art, 0.51 to 0.53 grams of ethanol are produced per gram of fermentable sugar, and this conversion factor can be used to calculate the theoretical ethanol that can be produced during a fermentation. Preferably, the fermentation is a simultaneous saccharification and fermentation (SSF). Conditions for fermentation using a yeast strain of the present invention are described below. The fermentable sugar content of a substrate, e.g., glucose and mannose, can be determined using methods known in the art. For instance, a substrate can be completely digested by exposure to heat and acidic conditions, and the presence of sugars separated using commercially available carbohydrate columns. Preferably, a yeast strain of the present invention has an ethanol yield of at least 80%, at least 85%, at least 90%, or at least 95% of the maximum theoretical yield. The percent of the maximum theoretical yield may be produced by a yeast of the present invention within 18 hours, within 24 hours, within 36 hours, within 48 hours, within 72 hours, within 96 hours, or within 120 hours of beginning the fermentation.

A yeast strain of the present invention may have the characteristic of fermenting a substrate within a set period of time. The time for fermenting a substrate may be measured as the time to deplete substrate or the time to produce a maximum amount of ethanol. The depletion of substrate present in a fermentation refers to depletion of the fermentable sugars to a concentration that is no greater than 10%, no greater than 8%, no greater than 6%, no greater than 4%, or no greater than 2% of the concentration of fermentable sugars present at the beginning of the fermentation. Preferably, the concentration of fermentable sugars present at the end of the fermentation is undetectable using methods known in the art.

A yeast strain has produced the maximum amount of ethanol for a particular fermentation when no more ethanol is produced by the fermentation. This may be measured by evaluating the amount of ethanol present at different times and comparing the amount of ethanol present at each time. Typically, the amount of ethanol is expressed as a concentration, such as grams of ethanol per liter of fermenter liquid volume (g/L), or as percent volume ethanol per volume of fermenter liquid volume (% v/v), or as percent weight ethanol per volume of fermenter liquid volume (% w/v). Ethanol yield may be expressed as yield per dry weight of starting material or yield per dry weight of fermentable sugars. A yeast strain of the present invention may produce a maximum amount of ethanol within 24 hours, within 36 hours, within 48 hours, within 72 hours, within 96 hours, or within 120 hours of beginning the fermentation. The time it takes for a yeast to produce a maximum amount of ethanol during a fermentation may be expressed as a percent of maximum. A yeast strain of the present invention may produce between at least 80% of maximum to at least 99% of maximum in single integer increments, i.e., at 80%, at least 81%, at least 82%, etc., up to at least 99% of maximum. The maximum amount of ethanol may be produced by a yeast of the present invention within 18 hours, within 24 hours, within 36 hours, within 48 hours, within 72 hours, within 96 hours, or within 120 hours of beginning the fermentation.

A yeast strain of the present invention may have the characteristic of fermenting a substrate to produce a maximum amount of ethanol. The maximum amount of ethanol produced may be at least 40 g/L, at least 42 g/L, at least 44 g/L, at least 46 g/L, at least 48 g/L, at least 50 g/L, or at least 52 g/L. The maximum amount of ethanol produced may be no greater than 66 g/L, no greater than 64 g/L, no greater than 62 g/L, no greater than 60 g/L, no greater than 58 g/L, no greater than 56 g/L, or no greater than 54 g/L.

A yeast strain of the present invention may have the characteristic of requiring less inoculum be added to a fermentation. Typically, amounts of yeast are added to fermentations to result in cell concentrations equivalent to between 2 and 5 grams dry yeast cells per liter (Rudolf et al., 2005, Enz. Microbial Technol., 37:195-204; Hoyer et al., 2009, J. Chem. Technol. Biotechnol., 84:570-577). A yeast strain of the present invention may be added to a fermentation in cell concentrations of between at least 0.02 and no greater than 2 grams yeast cells per liter, preferably at least 0.2 grams yeast cells per liter. Yeast cells may be lyophilized (dehydrated, freeze dried) and added on a weight basis to a known volume of fermentation, or cells may be monitored for growth using optical density as an indicator, or cell counts may be performed using a microscope and calibrated instrument such as a hemocytometer. Typically, 2 grams of yeast cells rehydrated in 1 liter of medium results in a cell count of 10⁸ cells using a hemocytometer. A typical inoculum level for the yeast strain described herein is 10⁶ cells, or 100 fold less cells than the minimum inoculum volume typically used.

A yeast strain of the present invention may have the characteristic of decreasing lag time. When a yeast strain is introduced into a fermentation medium the inoculated yeast pass through a number of stages. The initial period is referred to as the “lag phase” and may be considered a period of slow growth. Pretreated softwood creates a number of inhibitory compounds that increase the lag phase of microbial growth. During the next phase, referred to as the “exponential phase,” the growth rate gradually increases. After a period of maximum growth the rate ceases and the fermenting organism enters “stationary phase.” After a further period of time the fermenting organism enters the “death phase” where the number of viable cells declines. When introduced into a fermentation medium, a yeast strain of the present invention has a lag time that is less than the lag time of the same yeast prior to acclimatization.

Whether a yeast has one or more of the characteristics described herein can be determined by adding 10⁶ cells to pretreated lignocellulosic material, preferably pine, prepared using the one step or two step process described above, at a concentration of 12% dry matter solids, 15 FPU cellulase per gram dry weight of solids, 60 U of cellobiase per gram dry weight of solids, TSB (15 grams Tryptone (Pancreatic Digest of Casein), 5 grams Soytone (Papaic Digest of Soybean Meal), and 5 grams Sodium Chloride per liter), and water to a final volume of 100 ml (pH adjusted to 5 and temperature set at 37° C.). Pretreated pine using SO₂ or H₂SO₄ usually contains a number of inhibitors including the following (concentrations in mg/L): 5-hydroxymethylfurfural (at least 800 for 2 step process, at least 1200 for 1 step process), furfural (at least 700 for 2 step process, at least 900 for 1 step process), vanillic acid (at least 7.5 for 2 step process and at least 12 for 1 step process), vanillin (at least 60 for 2 step process and at least 20 for one step process), levulinic acid (at least 50 for two step process and at least 130 for one step process), and acetic acid (at least 500 for 2 step process and at least 900 for one step process). If the lignocellulosic material used is pretreated with the two step process, at least 30 grams ethanol/liter are produced in the first 24 hours, at least 44 grams ethanol/liter are produced in the first 48 hours, and the amount of ethanol produced at 48 hours is at least 98% of maximum If the lignocellulosic material used is pretreated with a one step process and a concentration of 15% dry matter solids is used (see Table 10), at least 19 grams ethanol/liter are produced in the first 24 hours, at least 34 grams ethanol/liter are produced in the first 48 hours, at least 39.2 grams ethanol/liter are produced in the first 72 hours, and at least 40 grams ethanol/liter are produced in the first 96 hours. If the yeast is frozen, preferably the yeast is revived as described herein before the characteristics of the yeast are determined.

An example of a yeast of the present invention is AJP50. AJP50 was deposited with American Type Culture Collection (ATCC), Manassas, Va., on May 8, 2009, and is designated ______. This deposit will be maintained under the terms of the Budapest Treaty on the International Recognition of the Deposit of Microorganisms for the Purposes of Patent Procedure. This deposit was made merely as a convenience for those of skill in the art and are not an admission that a deposit is required under 35 U.S.C. §112.

The present invention includes processes for fermenting lignocellulosic material into fermentation products using yeast having one or more of the characteristics as described herein. Published softwood fermentations (see, e.g., Rudolf et al., 2005, Enz. Microbial Technol., 37:195-204; Hoyer et al., 2009, J. Chem. Technol. Biotechnol., 84:570-577; Alkasrawi et al., 2006, Enzyme Microb. Tech., 38:279) typically use plating yeast on agar, inoculating a small flask (e.g., 300 ml) containing media (for example: (NH4)₂SO4, 7.5-10.8 g/L; KH₂PO₄, 3.5-5.0 g/L; MgSO₄, 0.75-1.1 g/L; trace metal solution, 10 ml/L; vitamin solution, 1 ml/L and glucose, 16.6-20.0 g/L) from a colony on the agar plate. The 300 ml seed flask may be grown overnight and used to produce enough yeast cells to inoculate a larger volume at 2-5 g/L concentration. For several processes (Rudolf et al., 2005, Enz. Microbial Technol., 37:195-204; Hoyer et al., 2009, J. Chem. Technol. Biotechnol., 84:570-577; Taherzadeh et al., 1997, Ind. Eng. Chem. Res., 36:4659) the seed inoculum ins then subjected to aerobic batch cultivation in a 2.5 L vessel with a defined medium containing glucose (e.g., (NH4)₂SO4, 20 g/L; KH₂PO₄, 9.4 g/L; MgSO₄, 2.0 g/L; trace metal solution, 26 ml/L; vitamin solution, 3 ml/L and glucose 17 g/L; Rudolf et al., 2005, Eng. Microbial Technol., 37:195-204). Then the pretreatment liquid (acid hydrolyzed wood liquid fraction) is slowly added to the fermenter and supplemented with glucose (Rudolf et al., 2005, Enz. Microbial Technol., 37:195-204; Hoyer et al., 2009, J. Chem. Technol. Biotechnol., 84:570-577). In some cases the solids from the acid pretreatment are washed to remove inhibitory compounds.

In contrast, cells of the present invention may be prepared for use in a fermentation using a smaller number of steps, such as reviving a frozen culture, growing it in a fermentation that includes, for example, between 6% and 8% solids. A composition that is used to revive a culture may be referred to herein as a reviving composition. In some aspects of the present invention, a culture revived by growth in, e.g., 6% and 8% solids may be used to directly inoculate a second fermentation that contains solids at a concentration of, for instance, 17.5%. Typically, this transfer from 6% and 8% solids to 17.5% solids may be useful when the pretreatment is the two step method described herein or another method the yields a similar level of toxic compounds. In other aspects of the present invention, a culture revived by growth in, e.g., 6% and 8% solids may be used to directly inoculate a second fermentation that contains solids at a concentration of, for instance, 12%, and this in turn may be used to directly inoculate a third fermentation that contains solids at a concentration of, for instance, 17.5%. Typically, this transfer from 6% and 8% solids to 17.5% solids using an intermediate step may be useful when the pretreatment is the one step method described herein or another method the yields a similar level of toxic compounds. These preparation steps may result in advantages that include using a lower number of yeast, result in yeast that are able to ferment in the presence of greater amounts of solids, and catalyze the fermentation in a shorter period.

Any suitable lignocellulosic material is contemplated in context of the present invention. Lignocellulosic material may be any material containing lignocellulose. In some aspects, the lignocellulosic material contains at least 50 wt %, preferably at least 70 wt %, more preferably at least 90 wt % lignocellulose. It is to be understood that the lignocellulose material may also include other constituents such as cellulosic material, such as cellulose, hemicellulose, and may also include constituents such as sugars, such as fermentable sugars and/or un-fermentable sugars.

Lignocellulosic material is generally found, for example, in the stems, leaves, hulls, husks, and cobs of plants or leaves, branches, and wood of trees. Lignocellulosic material can also be, but is not limited to, herbaceous material, agricultural residues, forestry residues, municipal solid wastes, waste paper, and pulp and paper mill residues. It is understood herein that lignocellulose material may be in the form of plant cell wall material containing lignin, cellulose, and hemicellulose in a mixed matrix. In some aspects the lignocellulosic material is corn fiber, rice straw, pine wood, poplar, wheat straw, switchgrass, bagasse, paper and pulp processing waste, corn stover, corn fiber, hardwood such as poplar and birch, softwood such as Douglas fir and pine and spruce, cereal straw, such as wheat straw, municipal solid waste, industrial organic waste, office paper, or mixtures thereof.

The process of producing fermentation products, preferably ethanol, from lignocellulosic materials typically includes pretreatment, enzyme hydrolysis, and fermentation. Detailed discussion of methods and protocols for the production of ethanol from biomass are reviewed in Wyman (1999, Annu. Rev. Energy Environ., 24:189-226), Gong et al. (1999, Adv. Biochem. Engng. Biotech., 65: 207-241), Sun and Cheng (2002, Bioresource Technol., 83:1-11), and Olsson and Hahn-Hagerdal (1996, Enzyme and Microb. Technol., 18:312-331).

There are numerous pretreatment methods or combinations of pretreatment methods known in the art and routinely used. Physical pretreatment breaks down the size of lignocellulosic material by milling or aqueous/steam processing. Chipping or grinding may be used to typically produce particles between 0.2 and 30 min in size. Methods used for lignocelluosic materials typically require intense physical pretreatments such as steam explosion. The most common chemical pretreatment methods used for lignocellulosic materials include dilute acid, alkaline, organic solvent, ammonia, sulfur dioxide, carbon dioxide or other chemicals to make the biomass more available to enzymes. Biological pretreatments are sometimes used in combination with chemical treatments to solubilize the lignin in order to make cellulose more accessible to hydrolysis and fermentation.

Steam explosion is a common method for pretreatment of lignocellulosic biomass and increases the amount of cellulose available for enzymatic hydrolysis (Foody, U.S. Pat. No. 4,461,648). Generally, the material is treated with high-pressure saturated steam and the pressure is rapidly reduced, causing the materials to undergo an explosive decompression. Steam explosion is typically initiated at a temperature of 160-260° C. for several seconds to several minutes at pressures of up to 4.5 to 5 MPa. The biomass is then exposed to atmospheric pressure. The process typically causes hemicellulose degradation and lignin transformation. Addition of H₂SO₄, SO₂, or CO₂ to the steam explosion reaction can improve subsequent cellulose hydrolysis, decrease production of inhibitory compounds and lead to the more complete removal of hemicellulose (Morjanoff and Gray, 1987, Biotechnol. Bioeng. 29:733-741).

In ammonia fiber explosion (AFEX) pretreatment, biomass is treated with approximately 1-2 kg ammonia per kg dry biomass for approximately 30 minutes at pressures of 1.5 to 2 MPa. (Dale, U.S. Pat. No. 4,600,590; Dale, U.S. Pat. No. 5,037,663; Mes-Hartree, et al. 1988, Appl. Microbiol. Biotechnol., 29:462-468). Like steam explosion, the pressure is then rapidly reduced to atmospheric levels, boiling the ammonia and exploding the lignocellulosic material. AFEX pretreatment appears to be especially effective for biomass with a relatively low lignin content, but not for biomass with high lignin content such as newspaper or aspen chips (Sun and Cheng, 2002, Bioresource Technol., 83:1-11).

Concentrated or dilute acids may also be used for pretreatment of lignocellulosic biomass. H₂SO₄ and HCl have been used at high concentrations, for instance, greater than 70%. In addition to pretreatment, concentrated acid may also be used for hydrolysis of cellulose (Hester et al., U.S. Pat. No. 5,972,118). Dilute acids can be used at either high (>160° C.) or low (<160° C.) temperatures, although high temperature is preferred for cellulose hydrolysis (Sun and Cheng, 2002, Bioresource Technol., 83:1-11). H₂SO₄ and HCl at concentrations of 0.3 to 2% (wt/wt) and treatment times ranging from minutes to 2 hours or longer can be used for dilute acid pretreatment.

Other pretreatments include alkaline hydrolysis (Qian et al., 2006, Appl. Biochem. Biotechnol., 134:273; Galbe and Zacchi, 2002, Appl. Microbiol. Biotechnol., 59:618), oxidative delignification, organosolv process (Pan et al., 2005, Biotechnol. Bioeng., 90:473; Pan et al., 2006, Biotechnol. Bioeng., 94:851; Pan et al., 2006, J. Agric. Food Chem., 54:5806; Pan et al., 2007, Appl. Biochem. Biotechnol., 137-140:367), or biological pretreatment.

Some of the pretreatment processes described, above include hydrolysis of the hemicellulose and cellulose to monomer sugars. Others, such as organosolv, prepare the substrates so that they will be susceptible to hydrolysis. This hydrolysis step can in fact be part of the fermentation process if simultaneous saccharification and fermentation (SSF) is used. Otherwise, the pretreatment may be followed by enzymatic hydrolysis with cellulases.

A cellulase may be any enzyme involved in the degradation of lignocellulose to glucose, xylose, mannose, galactose, and arabinose. The cellulolytic enzyme may be a multicomponent enzyme preparation, e.g., cellulase, a monocomponent enzyme preparation, e.g., endoglucanase, cellobiohydrolase, glucohydrolase, beta-glucosidase, or a combination of multicomponent and monocomponent enzymes. The cellulolytic enzymes may have activity, i.e., hydrolyze cellulose, either in the acid, neutral, or alkaline pH-range.

A cellulase may be of fungal or bacterial origin, which may be obtainable or isolated and purified from microorganisms which are known to be capable of producing cellulolytic enzymes, e.g., species of Humicola, Coprinus, Thielavia, Fusarium, Myceliophthora, Acremonium, Cephalosporium, Scytalidium, Penicillium or Aspergillus (see, for example, EP 458162), especially those produced by a strain selected from the species Humicola insolens (reclassified as Scytalidium thermophilum, see for example, Barbesgaard et al., U.S. Pat. No. 4,435,307), Coprinus cinereus, Fusarium oxysporum, Myceliophthora thermophila, Meripilus giganteus, Thielavia terrestris, Acremonium sp., Acremonium persicinum, Acremonium acremonium, Acremonium brachypenium, Acremonium dichromosporum, Acremonium obclavatum, Acremonium pinkertoniae, Acremonium roseogriseum, Acremonium incoloratum, and Acremonium furatum; preferably from the species Humicola insolens DSM 1800, Fusarium oxysporum DSM 2672, Myceliophthora thermophila CBS117.65, Cephalosporium sp. RYM-202, Acremonium sp. CBS 478.94, Acremonium sp. CBS 265.95, Acremonium persicinum CBS 169.65, Acremonium acremonium AHU 9519, Cephalosporium sp. CBS 535.71, Acremonium brachypenium CBS 866.73, Acremonium dichromosporum CBS 683.73, Acremonium obclavatum CBS 311.74, Acremonium pinkertoniae CBS157.70, Acremonium roseogriseum CBS134.56, Acremonium incoloratum CBS146.62, and Acremonium furatum CBS 299.70H. Cellulolytic enzymes may also be obtained from Trichoderma (particularly Trichoderma viride, Trichoderma reesei, and Trichoderma koningii), alkalophilic Bacillus (see, for example, Horikoshi et al., U.S. Pat. No. 3,844,890 and EP 458162), and Streptomyces (see, for example, EP 458162). Useful cellulases may be produced by fermentation of the above-noted microbial strains on a nutrient medium containing suitable carbon and nitrogen sources and inorganic salts, using procedures known in the art.

Examples of cellulases suitable for use in the present invention include, for example, CELLUCLAST (available from Novozymes A/S) and NOVOZYME (available from Novozymes A/S). Other commercially available preparations including cellulase which may be used include CELLUZYME, CEREFLO and ULTRAFLO (Novozymes A/S), LAMINEX and SPEZYME CP (Genencor Int.), and ROHAMENT 7069 W (Rohm GmbH). The cellulase enzymes are added in amounts effective from 5 to 35 filter paper units (FPU) of activity per gram of substrate, or 0.001% to 5.0% wt. of solids. Reaction conditions for enzymatic hydrolysis are typically around pH 4.8 at a temperature between 45 and 50° C. with incubations of between 10 and 120 hours. Surfactants may also be used during enzyme hydrolysis to improve cellulose conversion (Vlasenko et al., U.S. Pat. No. 7,354,743). Additionally, combinations or mixtures of available cellulases and other enzymes may also lead to increased saccharification.

Ethanol fermentation is the biological process by which sugars such as glucose are converted into cellular energy, thereby producing ethanol and carbon dioxide as metabolic waste products. Yeasts carry out ethanol fermentation on sugars in the absence of oxygen. Since the process does not require oxygen, ethanol fermentation is classified as anaerobic. The process begins with a molecule of glucose being broken down by the process of glycolysis into pyruvate. This reaction is accompanied by the reduction of two molecules of NAD⁺ to NADH and a net of two ADP molecules converted to two ATP plus the two water molecules. Pyruvate is then converted to acetaldehyde and carbon dioxide. The acetaldehyde is subsequently reduced to ethanol by the NADH from the previous glycolysis, which is returned to NAD⁺. For maximum efficiencies, both pentose sugars from the hemicellulose fraction of the lignocellulosic material (e.g. xylose) and hexose sugars from the cellulose fraction (e.g. glucose) can be used. Saccharomyces cerevisiae are widely used for fermentation of hexose sugars. Pentose sugars, released from the hemicellulose portion of the biomass, may be fermented using genetically engineered bacteria, including Escherichia coli (Ingram et al., U.S. Pat. No. 5,000,000) or Zymomonas mobilis (Zhang et al., 1995, Science, 267:240-243). Fermentation with yeast strains is typically optimal around temperatures of 30 to 37° C.

The steps following pretreatment, i.e. hydrolysis and fermentation, can be performed separately or simultaneously. Conventional methods used to process the lignocellulosic material in accordance with the methods of the present invention are well understood to those skilled in the art. The methods of the present invention may be implemented using any conventional biomass processing apparatus configured to operate in accordance with the invention.

Such an apparatus may include a batch-stirred reactor, a continuous flow stirred reactor with ultrafiltration, a continuous plug-flow column reactor (Gusakov, A. V., and Sinitsyn, A. P., 1985, Enz. Microb. Technol., 7: 346-352), an attrition reactor (Ryu, S. K., and Lee, J. M., 1983, Biotechnol. Bioeng., 25: 53-65), or a reactor with intensive stirring induced by an electromagnetic field (Gusakov, A. V., Sinitsyn, A. P., Davydkin, I. Y., Davydkin, V. Y., Protas, 0. V., 1996, Appl. Biochem. Biotechnol., 56: 141-153).

The conventional methods include, but are not limited to, saccharification, fermentation, separate hydrolysis and fermentation (SHF), simultaneous saccharification and fermentation (SSF), simultaneous saccharification and cofermentation (SSCF), hybrid hydrolysis and fermentation (HHF), and direct microbial conversion (DMC). The fermentation can be carried out by batch fermentation or by fed-batch fermentation.

SHF uses separate process steps to first enzymatically hydrolyze cellulose to glucose and then ferment glucose to ethanol. In SSF, the enzymatic hydrolysis of cellulose and the fermentation of glucose to ethanol are combined in one step (Philippidis, G. P., 1996, Cellulose bioconversion technology, in Handbook on Bioethanol: Production and Utilization, Wyman, C. E., ed., Taylor & Francis, Washington, D.C., 179-212). SSCF includes the coferementation of multiple sugars (Sheehan, J., and Himmel, M., 1999, Enzymes, energy and the environment: A strategic perspective on the U.S. Department of Energy's research and development activities for bioethanol, Biotechnol. Prog., 15: 817-827). HHF includes two separate steps carried out in the same reactor but at different temperatures, i.e., high temperature enzymatic saccharification followed by SSF at a lower temperature that the fermentation strain can tolerate. DMC combines all three processes (cellulase production, cellulose hydrolysis, and fermentation) in one step (Lynd, L. R., Weimer, P. J., van Zyl, W. H., and Pretorius, I. S., 2002, Microbiol. Mol. Biol. Reviews, 66: 506-577).

The final step may be recovery of the product. The fermentation or SSF product may be distilled using conventional methods producing ethanol, for instance 95% ethanol. For example, after fermentation the fermentation product, e.g., ethanol, may be separated from the fermented slurry. The slurry may be distilled to extract the ethanol, or the ethanol may be extracted from the fermented slurry by micro or membrane filtration techniques. Alternatively the fermentation product may be recovered by stripping. Methods for recovery are known in the art and used routinely.

The present invention is illustrated by the following examples. It is to be understood that the particular examples, materials, amounts, and procedures are to be interpreted broadly in accordance with the scope and spirit of the invention as set forth herein.

### Example 1

### Sample Description and Pretreatment

Loblolly pine from Georgia, USA, was chipped to a particle size of 10 mm or less. Chips were then pretreated with gaseous sulfur dioxide in one step (referred to as G4 below) or two steps (referred to as G3S2 below). G4 was obtained by treating a known amount of chips with 3% SO₂ at a temperature of 215° C. for 5 minutes and resulted in a single mixture, with liquid and solid mixed together.

G3 was obtained via a two-step process. A separate batch of a known amount of chips was treated with 2.5% SO₂ wt/wt of moisture content in chips, at a temperature of 190° C. for 5 minutes. Following this pretreatment step, the material was pressed using a hydraulic press to collect liquid. This liquid was called G3L1 and was not used in the experiments described herein. The pretreated solids (material remaining after the liquid was pressed out and removed), was then washed with water and pressed to a dry matter content of 40%. These washed dry matter solids are now called G3S1. G3S1 was used in the experiments described below.

In the second step, G3S described above (the washed solids pressed to a dry matter content of 40%) was impregnated with 2.5% SO₂ wt/wt of moisture content in the solids, and allowed to react at a temperature of 210° C. for 5 minutes. The samples obtained using these two steps of pretreatment were named G3L2 (L is for the liquid stream) and G3S2 (S is for the solids stream). G4 was obtained by treating a known amount of chips with 3% SO₂ at a temperature of 215° C. for 5 minutes and resulted in a single stream, with the liquid and solids remaining together. There was no washing of the solids for G4 and the conditions used were harsher (higher temperature and SO₂ concentration) than those used to produce G3.

### Compositional Analysis:

At the end of the pre-treatment regime, one step for G4, and at the end of the second step for G3, a sample of the digested wood slurry was removed and completely digested with heat and additional acid to monomeric sugars. Samples were analysed by HPLC (Shimadzu, Kyoto, Japan) equipped with a refractive index detector (Shimadzu, Kyoto, Japan). Glucose, mannose, arabinose, galactose and xylose were separated using an Aminex HPX-87P column (Bio-Rad, Hercules, USA) at 80° C., using water as eluent, at a flow rate of 0.5 mL/minute. Cellobiose, glucose, arabinose, lactic acid, glycerol, acetic acid, ethanol, HMF and furfural were separated on an Aminex HPX-87H column (Bio-Rad, Hercules, USA) at 65° C. using 5 mmol/L H₂SO₄ as the eluent, at a flow rate of 0.5 mL/minute. All samples were filtered through a 0.20 μm filter before HPLC analysis.

TABLE 1
Results of Compositional Analysis
(percent of each component on a dry weight basis):

| Sample | Glucan | Xylan | Galactan | Arabinan | Mannan | ASL | AIL | Sum |
|---|---|---|---|---|---|---|---|---|
| Fresh | 42.7 | 6.0 | 2.5 | 1.1 | 12.9 | 0.5 | 31.2 | 96.9 |
| Pine | | | | | | | | |
| G3 S2 | 53.9 | 1.9 | 0 | 0 | 0.5 | 0.4 | 48.7 | 105.4 |
| G4 | 54.5 | 1.2 | 0 | 0 | 0.4 | 0.5 | 48.9 | 105.5 |

(ASL: Acid-soluble lignin; AIL: Acid-insoluble lignin)

Simultaneous Saccharification and Fermentation (SSF) with an Increasing Solids Load: G4

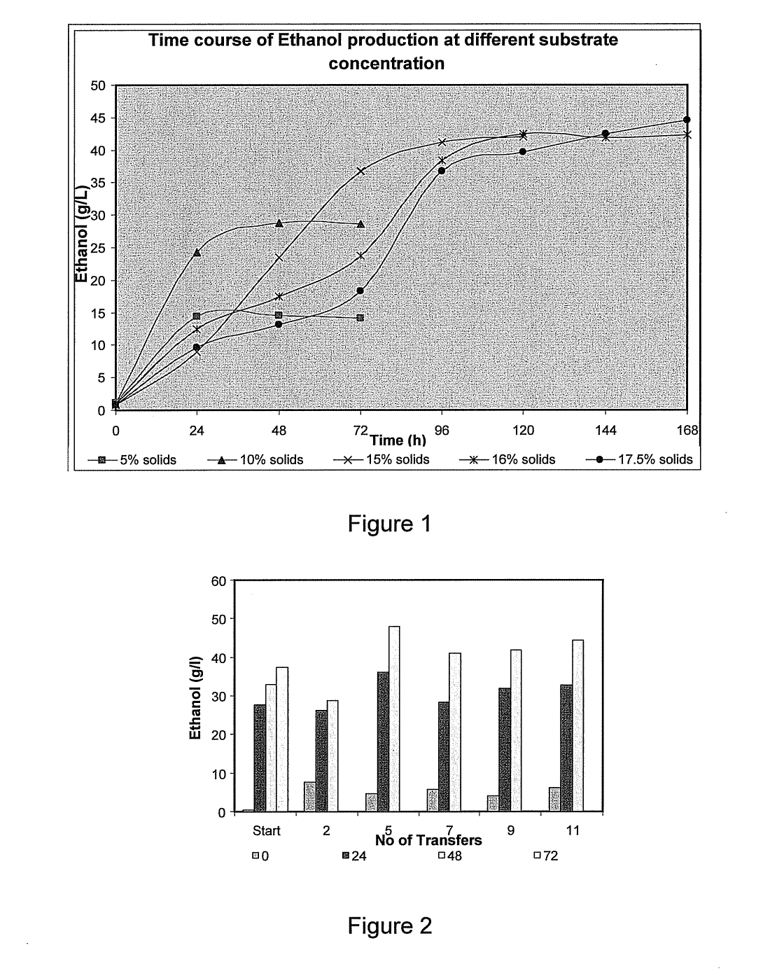

The pretreated loblolly pine sample, G4, was subjected to simultaneous saccharification and fermentation (SSF) using Saccharomyces yeast in 5 different sets with increasing concentrations of G4. Enzymes Novozyme 13 (a mixture of cellulases, Novozymes, Franklinton, N.C.) and Cellobiase (Novozymes, Franklinton, N.C.) were added at a concentration of 15 Filter Paper Units (FPU)/g dry weight of solids and 60 International Units (IU)/g dry weight of solids and active dried yeast (ADY) was added at a concentration of 2 g ADY/L. Saccharomyces cerevisiae BioFerm XR from North American Bioproducts (NABC, Norcross, Ga.) was used in these studies. Temperature of 37° C. and pH of 5 was maintained throughout the fermentation. The fermenter volumes were approximately 200 ml. Samples were collected every 24 hours and the amount of ethanol produced was estimated using Gas Chromatography (GC). Results of this study are summarized below in Table 2 and FIG. 1. As shown in FIG. 1, G4 pine pretreated in a one-step process (3% SO₂, 215° C., 5 min reaction time) demonstrates the decrease in fermentation rate with an increase in solids concentration. At 5 and 10% solids concentrations maximum ethanol concentrations were reached by 48 hours of fermentation. At 15%, 16% and 17.5% solids concentration an observed lag occurs between 24 and 48 hours of fermentation. Even though there was more substrate available, the amount of ethanol produced by 48 hours was 81.8%, 60.7%, and 45.6% of the ethanol produced by fermentation of the 10% solids, for 15%, 16%, and 17.5% solids concentrations, respectively. Maximum ethanol production was not obtained for the 15 and 16% solids fermentations until 120 hours of fermentation and 168 hours of fermentation for the 17.5% solids loading.

TABLE 2
Effect of substrate concentration on the production of Ethanol
(g/L) using G4 as substrate:

| | Substrate concentration (% dry weight solids per total volume) | | | | |
|---|---|---|---|---|---|
| Time (h) | 5% | 10% | 15% | 16% | 17.5% |
| 24 | 14.35 | 24.35 | 8.97 | 12.39 | 9.66 |
| 48 | 14.59 | 28.74 | 23.51 | 17.45 | 13.09 |
| 72 | 14.15 | 28.65 | 36.78 | 23.71 | 18.31 |
| 96 | | | 41.24 | 38.43 | 36.68 |
| 120 | | | 41.99 | 42.39 | 39.72 |
| 144 | | | | 41.91 | 42.41 |
| 168 | | | | 42.35 | 44.54 |

TABLE 3
Comparison of maximum yield (g ethanol/g of substrate)
obtained and time to reach maximum ethanol
production at different solids concentrations:

| Substrate | Maximum | Maximum yield | |
|---|---|---|---|
| Concentration (%) | Ethanol (g/L) | (g/g of substrate) | Time (h) |
| 5 | 14.59 | 0.29 | 48 |
| 10 | 28.74 | 0.29 | 48 |
| 15 | 41.99 | 0.28 | 120 (98% at 96 h) |
| 16 | 42.39 | 0.28 | 120 |
| 17.5 | 44.54 | 0.27 | 168 |

The results illustrate the lag in the production of ethanol with the increase in solid concentration. Though more ethanol is produced with a higher solids loading, the time to reach this maximum ethanol concentration is much longer.

A G4 sample was analyzed by HPLC mass spectroscopy (MS) for potential inhibitors, and high concentrations of toxic compounds such as 5-hydroxy-2-methylfurfural (1420.63 mg/L) and furfural compounds (957.41 mg/L) were measured. The yeast was affected by the toxicity and hence the yeast growth rate and ethanol production were hampered. The higher the solids content the more pronounced the lag phase. The fermenter is stirred and warmed, using energy while the yeast adapts to the fermentation conditions while slowly producing ethanol. To get the maximum ethanol production in a shorter timeframe from a high solids concentration and to decrease costs associated with the use of higher solids loading, we attempted to reduce this lag.

### Example 2

### Acclimatization of Yeast in Pretreated G4 Pine Solids

To reduce the lag phase observed in the production of ethanol with increasing solids, a process of acclimatization of yeast was introduced. Acclimatization of yeast is a process in which the yeast cells are in constant phase of growth in fresh solids so that they are forced to adapt to the toxic conditions present in the solids. Various regimes were examined including using the material from one fermentation to inoculate the next fermentation. Transfers made once every week, once every 96 hours, once every 72 hours, and once every 48 hours were examined. One set of results are described below.

BioFermXR was rehydrated from ADY in YP2D broth containing 10 g yeast extract, 20 g peptone, and 40 g dextrose, per liter dH₂O at 35° C. with stirring at 150 rpm. A sample of the yeast culture was plated onto tryptic soy agar and spread for isolated colonies. Individual yeast colonies were selected and used to inoculate YP2D broth (5-15 ml each) and incubated overnight at 35° C. with stirring at 150 rpm. The optical density at 600 nm (OD₆₀₀) was measured and the amount of culture required for an inoculum of an OD of 0.5 in the 100 ml of flask media was centrifuged. Culture supernatant was removed and the yeast pellet resuspended in 1× diluent. The washed cells were centrifuged again to form a cell paste and this paste was added to the fermenter volume for an initial inoculum level of an OD of 0.5. For this series of experiments, 17.5% dry weight G4 was autoclaved in a flask at 121° C. for 20 minutes. After cooling, G4 was treated with enzymes Novozyme 13 (15 FPU/g) and Cellobiase (60 IU/g) and 25 ml of 4× tryptic soy broth (TSB) and the cell paste was added at the same time as the enzymes for SSF. Fermentation was conducted at 37° C. at a pH of 5. Every 24 hours, a sample was drawn and ethanol was estimated by GC.

Generations 2-4. At time 168 h (168 hours after first inoculation of the flask), 44.5 g/L concentration of ethanol was obtained. This was the maximum value for ethanol in the 17.5% w/v solids fermentations. A 10% v/v inoculum from this fermentation was transferred to a fresh flask containing 17.5% solids, media, and enzymes as described previously. Samples were again drawn at every 24 hours and ethanol was estimated by GC. There was no ethanol production in the newly inoculated flask at 96 hours, only carryover of ethanol from the inoculum. Therefore, additional yeast equal to a dry weight content of 2 g/L was added to the same flask at 96 hours and allowed to ferment an additional 72 hours. The new yeast inoculum provided a jump-start for the fermentation and ethanol was produced. Again at 96 hours after addition of yeast a 10% inoculum was removed and added to a fresh flask of 17.5% w/v solids.

Generations 5-8. Ninety six hours was too long to wait before removing cells for transfer because they were not able to recover and grow in the high solids media at 17.5%. If these same cells were inoculated into lower solids concentrations, they were able to grow and produce ethanol, so at 72 hours after the re-inoculation, a 10% volume transfer was made to a fresh flask of 17.5% G4 solids (Table 4, D32). Repeated transfers after 72 hours of fermentation of 17.5% G4 solids were no more successful than the 96 hour trials Table 4, D46). The fermentation time of yeast in 17.5% G4 solids was reduced and a transfer was made at every 48 hours (Table 4, D52 and D54).

TABLE 4
Effect of yeast acclimatization on ethanol production from
G4 pine at 17.5% dry weight solids treated with enzymes
Novozyme 13 and Cellobiase at a conc. of 15 FPU/g and 60 U/g.

| | Date of transfer (10% inoculum) and ethanol | | | | |
|---|---|---|---|---|---|
| | production (g/l) | | | | |
| | Start | | | | |
| Time (h) | (D0) | D32 | D46 | D52 | D54 |
| 0 | 0.98 | 5.16 | 3.56 | 2.67 | 3.63 |
| 24 | 5.10 | 5.32 | 2.9 | 14.65 | 16.25 |
| 48 | 13.09 | 4.9 | 2.85 | 33.78 | 33.13 |
| 72 | 28.31 | 4.8 | 2.85 | | 39.72 |
| 96 | 36.80 | | | | 44.55 |
| 120 | 39.72 | | | | |
| 144 | 42.41 | | | | |
| 168 | 44.58 | | | | |
| 24* | | 32.88 | 22.71 | | |
| 48* | | 47.93 | 26.02 | | |
| 72* | | 44.16 | 26.89 | | |

*Time in h after yeast was added. D0, D32, D46, D52 and D54 refer to day 0, day 32, day 46, day 52 and day 54, respectively.

Reduction in lag in the production of ethanol can be seen in D52 and D54 when a transfer of 10% inoculum was made from the previous flask at time 48 hours. This represents successful acclimatization of yeast in 17.5% solids pretreated G4 pine fermentations. From D32 to D46, there was no additional ethanol produced up to 72 hours. From D52, there was 33.78 g/L ethanol produced by 48 hours, instead of just 2.85-13.09 g/L in the previous fermentations. On D54 a 10% inoculum was removed and added to a separate flask, this time instead of producing little or no ethanol, the fermentation reached 35 g/L ethanol by 48 hours. At 96 hours where a maximum of 44 g/L ethanol were obtained, and at 168 h 49.5 g ethanol/L was present. Previously it took 168 hours to reach 44.5 g ethanol/L concentration of ethanol and it had not been possible to reach 49.5 g ethanol/L.

### Example 3

### Acclimatization of Yeast in Pretreated G3S2 Pine Solids from a Two-Step Pretreatment Process

G3S2 was also analyzed by HPLC MS for inhibitor content. G3 was determined to be less toxic than G4, containing 856.44 mg/l of 5-hydroxy-2-methylfurfural and 738.26 mg/l of furfural. Due to the presence of these toxic compounds, a less pronounced lag was observed in the production of ethanol when fermentations were carried out with yeast at 17.5% pretreated G3S2 load.

To reduce the lag in the production of ethanol, yeast was allowed to acclimatize in 17.5% pretreated G3S2. G3S2 (17.5% dry weight) were autoclaved in a flask at 121° C. for 20 minutes. After cooling, G3S2 was treated with enzymes Novozyme 13 and Cellobiase at a concentration of 15 FPU/g and 60 IU/g. Twenty five milliliters of 4× TSB was added. BioFermXR was rehydrated from ADY in YP2D broth containing 10 g yeast extract, 20 g peptone, 40 g dextrose, per liter dH₂O at 35° C. with stirring at 150 rpm. A sample of the yeast culture was plated onto tryptic soy agar and spread for isolated colonies. Individual yeast colonies were selected and used to inoculate YP2D broth (5-15 ml each) and incubated overnight at 35° C. with stirring at 150 rpm. The optical density at 600 nm (OD₆₀₀) was measured and the amount of culture required for an inoculum of an OD of 0.5 in the 100 ml of flask media was centrifuged. Culture supernatant was removed and the yeast pellet resuspended in 1× diluent. The washed cells were centrifuged again to form a cell paste and this paste was added to the fermenter volume for an initial inoculum level of an OD of 0.5. Enzymes and yeast inoculum were added at the same time for SSF.

Fermentation was carried out at 37° C. at a pH of 5. Every 24 hours a sample was drawn and ethanol was estimated by GC. At time 72 hours, a 10% v/v inoculum from this flask was transferred to a fresh flask with 17.5% solids, media and enzymes required for the same. Ethanol was measured every 24 hours. Five transfers were made after every 72 hours or more of fermentation, then 48 hour transfers were made for the remainder of the study. A decrease in the lag phase of ethanol production was observed at 48 hours of each transfer made thereafter (Table 5).

TABLE 5
Time course of acclimatization of yeast and ethanol production (g/L) in 17.5% G3S2 and
enzymes (Novozyme 13 and Cellobiase at 15 FPU/g and 60 U/g):

| Time | No. of Transfers | | | | | | | | | | | |
|---|---|---|---|---|---|---|---|---|---|---|---|---|
| (h) | Start | 1 | 2 | 3 | 4 | 5 | 6 | 7 | 8 | 9 | 10 | 11 |
| 0 | 0.49 | | 7.6 | 4.66 | 4.85 | 4.61 | 5.75 | 5.75 | 5.82 | 4.02 | 4.5 | 6.2 |
| 24 | 27.6 | 12.4 | 26.1 | 12.1 | 27.5 | 36.1 | 27.4 | 28.4 | 31.6 | 31.9 | 28.4 | 32.8 |
| 48 | 32.9 | 50.2 | 28.7 | 30.8 | 33.6 | 48.0 | 35.2 | 40.9 | 35.0 | 41.9 | 41.2 | 44.4 |
| 72 | 37.4 | 45.9 | 30.5 | 27.8 | 32.9 | | | | | | | |

‘Start’ in Table 5 and FIG. 2 represents the flask in which yeast was added to begin the acclimatization process. Transfer of 10% v/v inoculum was made at time 72 h from the start flask to a flask with fresh 17.5% G3 solids. Numbers 2, 5, 7, 9, 11 represents respective transfers made with 10% inoculum from previous flasks. Reduction in lag in the production of ethanol can be seen in transfers 5 onwards with an increase in the production of ethanol at time 48 h. This represents the successful acclimatization of yeast in 17.5% pretreated G3 pine fermentations treated with enzymes Novozyme 13 and Cellobiase at 15 FPU/g and 60 IU/g dry weight of G3 solids.

### Example 4

### Storage and Revival of Acclimatized Yeast for Fermentation of 17.5% Dry Weight G3S2

Glycerol stocks containing acclimatized yeast from earlier high solids fermentations were thawed and a 0.5 ml glycerol stock (40% w/v) was added to each of 3 different flasks with 50 ml TSB and varying concentration of G3S2 (2%, 5%, and 7% dry weight total solids) and adjusted to a pH of 5.0. Each flask also received the equivalent of 15 FPU cellulase and 60 U cellobiase/g dry wt total solids for SSF. Yeast cells were added directly after enzyme addition for SSF. Flasks were incubated at 37° C. for 48 h with shaking at 150 rpm. Growth of yeast was checked in each flask via direct counts and plating onto tryptic soy medium containing 7% dry weight G3S2. The growth of yeast directly from frozen glycerol stocks to liquid culture containing over 2% concentrations of G3S2 solids was expected to be non-existent, or very slow at best. Surprisingly, cell counts reached 5×10⁸ colony forming units/ml of culture. Also, fermentations were set up in 2 flasks (total volume 50 ml) containing 17.5% dry weight G3S2 solids, one flask was inoculated with 10% inoculum of yeast grown in TSB broth with 7% dry weight G3S2 solids and the other flask was inoculated with 2 g/L active dried yeast (ADY). Novozyme 13 and Cellobiase were added at a concentration of 15 FPU/g dry weight solids and 60 U/g dry weight solids. Samples were collected every 24 h for ethanol estimation.

TABLE 6
Ethanol production (g/L) during SSF by non-acclimatized
and AJP50 in 17.5% G3S2.

| | | 10% inoculum from TSB |
|---|---|---|
| Time (h) | ADY (2 g/L) | flask with 7% GA3 solids |
| 0 | 0.5 | 2.3 |
| 24 | 27.3 | 31.05 |
| 48 | 37.6 | 45.25 |

Conclusion: The acclimatized yeast reached 45.25 g ethanol/L by 48 hours of fermentation. Using ADY under the same conditions resulted in a much lower ethanol yield of 37.6 g/L.

### Example 5

### Storage and Revival of Acclimatized Yeast for Fermentation of 17.5% Solids

A typical inoculation regime for AJP 50 yeast is described. First a glycerol (40% w/v) stock culture is thawed from the freezer and added to a flask containing 7% pretreated pine solids on a dry weight basis. The glycerol stock contains approximately 10⁶ cells. The flask contains unwashed pretreated pine solids at a concentration of 7% dry matter that is autoclaved, then tryptic soy broth (TSB) is added and the pH adjusted to 5.0. Cellulases are added at 15 filter paper units (FPU)/g dry wt of solids and cellobiase or beta-glucosidase at 60 cellobiase units/g dry wt of solids and the temperature adjusted to 37° C. One vial of frozen glycerol stock yeast is added to 9.2 ml TSB and cell count taken via hemocytometer and microscope (routinely 10⁶ cells). The cells now suspended in 10 ml TSB are then added to the flask containing the pretreated solids, TSB, and enzymes for SSF. Samples are removed at times (T) 0 hour (right after inoculation), 24 hours, 36 hours, and 48 hours. Ethanol is usually obtained between 24 and 48 hrs from the glycerol stock inoculum. Fermentation is routinely conducted for 24-48 hours in 7% solids before transferring to a higher solids fermentation.

### Example 6

### Comparison of Acclimatized Yeast with Parental Yeast and Fermentation of Solids

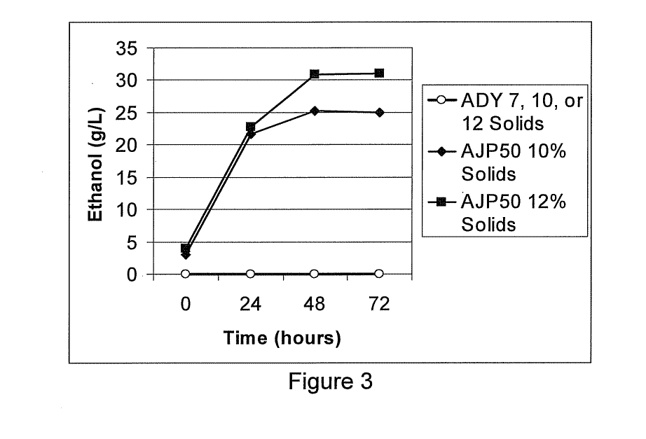

Rehydration of 0.2 grams active dry yeast (ADY) in tryptic soy broth (TSB) results in a cell count of 10⁸ cells. If added to 100 ml this would reflect the minimum inoculum typically used in previous studies from other laboratories (2 g/L). This was diluted 100× to reflect the same amount of cells as were frozen in glycerol stocks for AJP50 (10⁶ cells). Flasks were prepared as described previously except the biomass was subjected to a higher SO₂ (3.3% versus 3.0% in G4 above) concentration at a slightly lower reactor temperature (213° C. versus 215° C. in G4 above) for the same 5 minute reaction time. When ethanol production was evaluated with gas chromatography every 24 hours, no ethanol was observed in the flasks containing ADY and 7% or 12% solids. Conversely in the AJP50 flasks, ethanol was produced routinely between 24 and 48 hours in the 7% solids, but not after 72 hours in the initial 12% solids flask. Using the flask containing AJP50 and 7% solids as the seed for inoculation of 10% and 12% solids flasks resulted in ethanol production at 24 hours for both the 10% and 12% solids flasks (FIG. 3).

Ethanol production in flasks described above using 3.3% SO₂ pretreated pine under pretreatment conditions of 213° C. for 5 minutes and 7% solids (1^{st} flask from glycerol stock 24 hours=4 grams ethanol/L and 48 hours=15 grams ethanol/L). Twenty ml (10% of the second fermenter volume) of the 7% solids fermentation was used to inoculate two sets of flasks at 10% solids and 12% dry matter solids. The resulting ethanol production is shown in Table 7.

TABLE 7
Ethanol (g/l):

| | AJP50 | |
|---|---|---|
| Time (h) | 10% | 12% |
| 0 | 4.5 | 4.0 |
| 24 | 21.7 | 22.8 |
| 48 | 25.3 | 30.8 |

### Example 7

### Acclimatized Yeast have Reduced Lag Phase

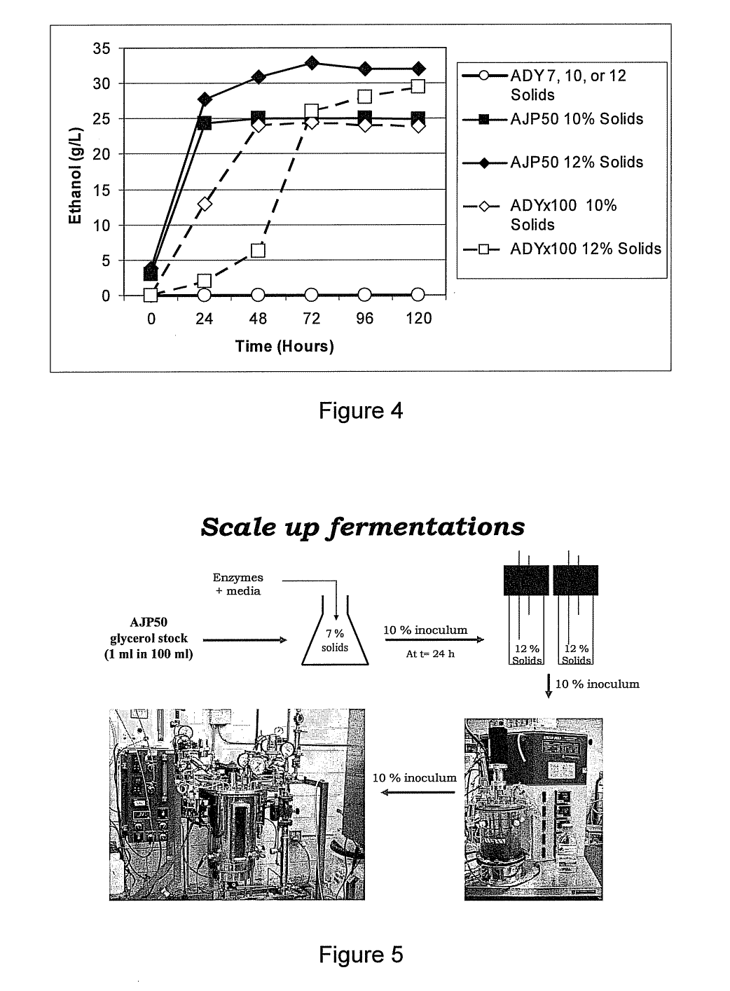

Using 10⁶ ADY cells to inoculate fermentations with 15 FPU cellulase/g dry wt pretreated pine (3.5% SO₂, 215° C., 5 minutes) and 60 U cellobiase/g dry wt pretreated pine, at 7, 10, and 12% solids, there is no ethanol produced after 120 hours (FIG. 4). When the inoculum is increased to 10⁸ cells or 2 g/L, ethanol is produced in 12% solids, but the ethanol production is slow, exhibiting a lag in product accumulation, reaching approximately 28.5 gram ethanol/L by 72 hours. In contrast AJP50 reached 28 grams ethanol/L by 24 hours of fermentation with no observable lag in ethanol production. Therefore it can be concluded that under these conditions AJP50 reduced the lag time by 48 hours. AJP50 produced 31 grams ethanol/L by 72 hours; the ADY parental strain was only at 28 grams/L by 72 hours and stopped at 29.5 g ethanol/L, just short of the 31 grams/L generated by AJP50. Yeast can metabolize the ethanol produced so it is not uncommon to see ethanol values decline over time when substrate is limiting. A comparison of ethanol production by AJP50 strain and parent ADY is presented in FIG. 4.

ADY fermentations at 7,10, or 12% solids are all represented by the open circles (◯) along the bottom of the graph in FIG. 4, as no ethanol was produced at any time point during the fermentation. When the inoculum for ADY was increased 100× to 10⁸ cells (□ and ⋄), then ethanol production increased. Using 100× more cells for inoculation at 10% solids resulted in 13 g/L ethanol at 24 hours and 24.5 g/L ethanol at 72 hours, just slightly slower than AJP50. At 12% solids with ADY there was a more pronounced lag phase with a significant increase in ethanol production between 48 and 72 hours; 21% of maximum ethanol was reached at 24 h, 88.9% of the maximum ethanol production reached by 72 hours, and 95% of the maximum ethanol production reached by 96 hours. For AJP50, 97.6% of the maximum ethanol was reached for 10% solids by 24 hours. At a solids content of 12%, AJP50 reaches its maximum ethanol production by 72 hours of 31 grams ethanol/L, which is essentially all of the ethanol that could be produced from this amount of pretreated pine. AJP50 reached 95% of maximum theoretical (and actual ethanol measured in the fermentation) by 48 hours, and 84% of maximum by 24 hours.

### Example 8

### Comparison of AJP50 Strain to Parent ADY in Various Pretreated Softwood Fermentations

Comparison of AJP50 strain to parent ADY in two step SO₂ pretreated pine fermentations (Table 8). All fermentations used 15 FPU cellulase/g dry wt of pretreated pine solids and 60 U cellobiase/g dry wt of pretreated pine solids added just after pH adjust to 5.0 and just prior to addition of yeast (SSF). All ethanol concentrations in grams/liter.

TABLE 8
Comparison of AJP50 strain to parent ADY in two step
SO₂ pretreated pine fermentations.

| Pretreatment Condition: | AJP 50 | ADY Parental Strain |
|---|---|---|
| 2 Step SO₂ | (0.02 g/L or 10⁶ cells) | (2 g/L or 10⁸ cells) |
| Step 1: T 190° C. | 17.5% | 17.5% |
| 5 min, 2.5% SO₂ | 24 h = 31.1 | 24 h = 26.3 |
| Step 2: T 210° C. | 48 h = 45.3 | 48 h = 35.6 |
| 5 min, 2.5% SO₂ | 72 h = 45.7 | 72 h = 39.2 |

Comparison of AJP50 strain to parent ADY in Single Step SO₂ pretreated pine fermentations (Table 9). All fermentations used 15 FPU cellulase per g dry wt of pretreated pine solids and 60 U cellobiase/g dry wt of pretreated pine solids. All ethanol concentrations in grams/liter.

TABLE 9
Comparison of AJP50 strain to parent ADY in Single Step SO₂ pretreated pine fermentations.

| Pretreatment | AJP 50 | | ADY Parental Strain | |
|---|---|---|---|---|
| G4 | 10⁶ Cells | | 10⁶ Cells | |
| T = 215 C., 5 min, | 7% Solids | | 7% Solids | |
| 3.0% SO₂ | 24 h = 10.5 | | 24 h = 0.9 | |
| Maximum | | | 48 = 3.0 | |
| Theoretical | 7% above for inoculation of 17.5% solids | | 7% above for inoculation of 12% solids; 2 g/L ADY | |
| yield = 50.9 g/L | | | revived in TSB for inoculum of 17.5% solids | |
| | 17.5% Solids | | 10⁶Cells | 10⁸ Cells |
| | 24 h = 22.7 | | 12% Solids | 17.5% Solids |
| | 48 h = 35.0* (70% of maximum theoretical) | | 24 h = 0.9 | 24 h = 9.7 |
| | 72 h = 42.5 (85% of maximum theoretical) | | 48 h = 2.8 | 48 h = 13.1 |
| | 96 h = 44.5 | | 72 h = 7.0 | 72 h = 18.3 |
| | 120 h = 49.3 | | 96 h = 7.2 | 96 h = 36.7 |
| | | | | 120 h = 39.7 |
| | *71% of the total ethanol obtained during | | | 144 h = 42.2 |
| | fermentation | | | 168 h = 44.5 |
| T = 213° C., 5 min, | 10⁶ Cells | | 10⁶ Cells | |
| 3.3% SO₂ | 7% Solids | | 7% Solids | |
| Maximum | 24 h = 4 | | 24 h = no ethanol | |
| Theoretical yield : | 48 h = 14.8 | | 48 h = no ethanol | |
| 10% solids = 26 g/L | 7% above for inoculation of 10 and 12% | | 2 g/L ADY revived in TSB for inoculum of 10 | |
| 12% solids = 31.2 g/L | solids | | and 12% solids | |
| | 10% Solids | 12% Solids | 10% Solids | 12% Solids |
| | 24 h = 21.7 | 24 h = 22.8 | 24 = 22.5 | 24 h = 8.4 |
| | 48 h = 25.3 | 48 h = 30.8 (98.7% of max theo) | 48 = 25.4 | 48 h = 21.0 |
| T = 215° C., 5 min, | 10⁶ Cells | | 10⁶ Cells | |
| 3.5% SO₂ | 7% Solids | | 7% Solids | |
| Maximu | 24 h = 6.0 | | 24 h = no ethanol | |
| Theoretical yields: | 48 h = 16.8 | | 48 h = no ethanol | |
| 10% solids = 25.44 | 7% for inoculation of 10% and 12% Solids | | Same inoculum level as AJP50 = no ethanol | |
| 12% solids = 31.0 | | | at 7, 10, 12% Solids concentration | |
| | | | 2 g/L ADY revived in TSB for inoculum of | |
| | | | 10 and 12% solids | |
| | 10% Solids | 12% Solids | 10% Solids | 12% Solids |
| | 24 h = 24.4 | 24 h = 26.0 | 24 h = 13 | 24 h = 2.0 |
| | 48 h = 25.1 | 48 h = 29.5* (95% of max) | 48 h = 24.3 | 48 h = 6.2* |
| | 72 h = 25.0 | 72 h = 31.0 | 72 h = 24.5 | 72 h = 26.0 |
| | | 96 h = 30.9 | | 96 h = 28.0 |
| | | 120 = 30.0 | | 120 = 29.5 |
| | | *48 h @ 95% of the total ethanol | | *48 h @ 21% of the total ethanol |
| | | obtained in the fermentation | | obtained in the fermentation |

TABLE 10
Increasing solids concentration in fermentations using AJP50 in Single Step SO₂ pretreated pine fermentations using
T = 213° C., 5 min, 2.9% SO₂. All fermentations were inoculated using 10⁶ Cells from a 7% solids fermentation
inoculated from glycerol freezer stocks. All values are presented in g/L.

| Time of | | | | |
|---|---|---|---|---|
| Fermentation | 12% Solids | 13% Solids | 14% Solids | 15% Solids |
| Sample | Maximum Theoretical = 31.8 | Maximum Theoretical = 35.0 | Maximum Theoretical = 37.1 | Maximum Theoretical = 40 |
| 24 h | 20.2 | 21.4 | 21.2 | 19.0 |
| 48 h | 30.3 (95% of max theo) | 31.5 (90% of max theo) | 31.8 (85.5% of max theo) | 34 (85% of max theo) |
| 72 h | 31.2 | 35.0 | 36 | 39.2 |
| 96 h | | | 36.9 | 40 |

### Example 9

### Scale Up Fermentations

One vial of glycerol stock (10⁶ AJP50 cells) was used to inoculate 7% solids in a flask of 100 ml, then 10% of the fermenter volume was added as inoculum for 12% solids in 200 ml, then 3 liter, and then 20 liter fermenters (FIG. 5). The enzyme loading for all was 15 FPU cellulase/g dry wt pretreated pine and 60 U cellobiase/g dry wt pretreated pine (215° C., 5 min, 3.5% SO₂). The flask containing 7% solids contained 6 grams ethanol/L at 24 hours fermentation. Twenty ml of the 7% fermentation was added to each of two 200 ml volume fermenters with 12% solids and ethanol concentration at 24 hours was 23.1 g ethanol/L. Three hundred ml from the 12% solids fermentation was used to inoculate a 3000 ml (3 L) 12% solids fermentation. Ethanol content at 24 hours was 8 g/L and at 48 hours it was 29.3 g/L. The 3 L fermentation was used to inoculate a 20 L fermenter with 12% solids. Ethanol concentrations were as follows: at 24 hours 25.62 grams ethanol/L; at 48 hours 28.2 grams ethanol/l; and at 72 hours 31.3 grams ethanol/L. The maximum theoretical ethanol yield from this particular pretreated pine sample was 32 g/l based on the fermentable sugar content (45% of the dry matter in the pretreated pine). Subtracting 2 g/L ethanol production from media components other than pine revealed approximately 80% of the theoretical maximum ethanol yield was obtained by 24 hours, 88% by 48 hours, and 91.5% at 72 hours.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A method for producing ethanol comprising:
fermenting a composition with a yeast, wherein the composition comprises pretreated solid lignocellulosic material, wherein the pretreated solid lignocellulosic material is present at a concentration of at least 12% solids, and wherein at least 30 grams ethanol per liter are produced.

2. The method of claim 1 wherein the at least 30 grams ethanol per liter are produced within 48 hours.

3. The method of claim 1 wherein at least 40 grams ethanol per liter are produced.

4. The method of claim 3 wherein the at least 40 grams ethanol per liter are produced within 48 hours.

5. (canceled)

6. The method of claim 1 wherein the composition comprises furfural, 5-hydroxymethylfurfural, formic acid, acetic acid, vanillic acid, vanillin, levulinic acid, or a combination thereof, wherein the concentration of furfural is at least 700 milligram per liter (mg/L), the concentration of 5-hydroxymethylfurfural is at least 800 mg/L, the concentration of formic acid is at least 200 mg/L, the concentration of acetic acid is at least 500 mg/L, the concentration of vanillic acid is at least 7.5 mg/L, the concentration of vanillin is at least 20 mg/L, and the concentration of levulinic acid is at least 50 mg/L.

7. (canceled)

8. (canceled)

9. The method of claim 1 wherein the pretreated solid lignocellulosic material is pine.

10. The method of claim 9 wherein the pine is Pinus taeda.

11. The method of claim 1 wherein prior to the fermenting the yeast is revived from a frozen stock, wherein the reviving comprises transferring a frozen stock directly to a reviving composition comprising the pretreated solid lignocellulosic material.

12. The method of claim 11 wherein the reviving composition the pretreated solid lignocellulosic material at a concentration of at least 6%.

13. (canceled)

14. The method of claim 1 wherein the pretreated solid lignocellulosic material is present in the reviving composition at a concentration of between 6% and 9%.

15. The method of claim 1 wherein the fermenting is a batch simultaneous saccharification and fermentation.

16. The method of claim 1 wherein the yeast is Saccharomyces cerevisiae.

17. The method of claim 16 wherein the Saccharomyces cerevisiae is AJP50.

18. (canceled)

19. A yeast that ferments a composition comprising pretreated solid lignocellulosic material at a concentration of at least 12% solids to yield at least 30 grams ethanol per liter in 48 hours.

20. The yeast of claim 19 wherein the pretreated solid lignocellulosic material is not detoxified.

21. The yeast of claim 19 wherein the composition comprises furfural, 5-hydroxymethylfurfural, formic acid, acetic acid, vanillic acid, vanillin, levulinic acid, or a combination thereof, wherein the concentration of furfural is at least 700 mg/L, the concentration of 5-hydroxymethylfurfural is at least 800 mg/L, the concentration of formic acid is at least 200 mg/L, the concentration of acetic acid is at least 500 mg/L, the concentration of vanillic acid is at least 7.5 mg/L, the concentration of vanillin is at least 20 mg/L, and the concentration of levulinic acid is at least 50 mg/L.

22. (canceled)

23. The yeast of claim 19 wherein the yield at least 40 grams ethanol per liter in 48 hours.

24. The yeast of claim 19 wherein the yeast comprises the characteristics of AJP50.

25. An isolated yeast AJP50 deposited with the American Type Culture Collection in accordance with the provisions of the Budapest Treaty.

26. A method for acclimatizing yeast comprising:
culturing yeast in a first composition comprising pretreated solid lignocellulosic material;
transferring a sample comprising the cultured yeast to a second composition comprising pretreated solid lignocellulosic material and culturing the yeast in the second composition;
repeating the transferring until the yeast will produce at least 30 grams ethanol per liter during a batch simultaneous saccharification and fermentation with at least 12% solids.

27. The method of claim 26 wherein the pretreated solid lignocellulosic material in the first composition is present at a concentration of at least 5% solids.

28. The method of claim 26 wherein the second composition comprises the pretreated solid lignocellulosic material at a concentration greater than the first composition.

29. The method of claim 26 wherein the repeating comprises transferring the yeast to compositions comprising the pretreated solid lignocellulosic material at increasing concentrations of solids.

30. The method of claim 26 wherein the pretreated solid lignocellulosic material is not detoxified prior to the culturing or the transferring.

31. The method of claim 26 wherein the first composition comprises furfural, 5-hydroxymethylfurfural, formic acid, acetic acid, vanillic acid, vanillin, levulinic acid, or a combination thereof, wherein the concentration of furfural is at least 700 mg/L, the concentration of 5-hydroxymethylfurfural is at least 800 mg/L, the concentration of formic acid is at least 200 mg/L, the concentration of acetic acid is at least 500 mg/L, the concentration of vanillic acid is at least 7.5 mg/L, the concentration of vanillin is at least 20 mg/L, and the concentration of levulinic acid is at least 50 mg/L.

32. (canceled)

33. The method of claim 26 wherein the pretreated solid lignocellulosic material is pine.

34. The method of claim 33 wherein the pine is Pinus taeda.

35. The method of claim 26 wherein the culturing comprises a simultaneous saccharification and fermentation.
